(19) United States
(12) Patent Application Publication
(10) Pub. No.: US 2019/0351118 A1
(43) Pub. Date: Nov. 21, 2019

(54) HEART PUMP, AND METHOD FOR OPERATING A HEART PUMP

(71) Applicant: Berlin Heart GmbH, Berlin (DE)

(72) Inventors: Kurt Graichen, Berlin (DE); Constantin Wiesener, Potsdam (DE); Sebastian Kallenbach, Kassel (DE)

(73) Assignee: Berlin Heart GmbH, Berlin (DE)

(21) Appl. No.: 15/754,998

(22) PCT Filed: Aug. 22, 2016
(86) PCT No.: PCT/EP2016/069833

§ 371 Date: Feb. 23, 2018

(30) Foreign Application Priority Data
Aug. 24, 2015 (EP) 15 182 128.7

(51) Int. Cl. A61M 1/12; A61M 1/10

(52) U.S. Cl. CPC A61M 1/122; A61M 1/1086; A61M 1/1008; A61M 2205/3368; A61M 2205/3334; A61M 2230/04; A61M 1/1075

(57) ABSTRACT

A heart pump and a method for operating a VAD (ventricular assist device) heart pump, in which method a time-dependent pressure of the blood to be delivered is measured directly by means of at least one pressure sensor arranged on the pump inlet and/or on the pump outlet. A temperature of the blood is additionally detected. By evaluating the measured pressure profiles and/or corresponding rates of change of the measured pressure, it is possible for delivery parameters of the pump, and physiological values of the heart assisted by the pump, to be determined in a convenient and reliable manner.

### CROSS-REFERENCE TO RELATED APPLICATIONS

This application is a 371 nationalization of international patent application PCT/EP2016/069833 filed Aug. 22, 2016, which claims priority under 35 USC § 119 to European patent application EP 15182128.7 filed on Aug. 24, 2015, both of which are hereby entirely incorporated by reference.

### TECHNICAL FIELD

The invention lies in the field of mechanical and electrical engineering and can be used particularly advantageously in the field of medical technology.

In particular, the invention relates to the operation of a heart pump.

### BACKGROUND

For some years, heart pumps for delivering blood and for replacing or assisting a patient's heart have been known. Pumps of this kind can be embodied in various forms and can be operated in different ways. They can essentially replace the patient's heart and take on the function thereof fully, or can also be used merely to support a heart that is not capable of performing its full function, wherein a residual cardiac activity takes place and is also to be supported.

### BRIEF DESCRIPTION OF THE DRAWINGS

FIG. 1 schematically shows an arrangement of a heart assistance pump in a patient's body;

FIG. 2 shows a detected pressure profile in the region of the pump;

FIG. 3 shows the intake end of a cannula connected to a heart assistance pump in a ventricle;

FIG. 4 shows a heart assistance pump with two pressure sensors;

FIG. 5 shows the basic functional elements of a typical heart assistance pump;

FIG. 6 shows three graphs of a model for determining the cardiac output on the basis of ventricular pressure measurements;

FIG. 7 shows a graph showing the development of ventricular pressure over two diastoles and an intermediate systole;

FIG. 8 schematically shows a blood pump having combined pressure and temperature sensors on each of an inlet and an outlet; and

FIG. 9 shows a schematic structure of a combined sensor.

### DETAILED DESCRIPTION

What are known as VADs (ventricular assist devices, hereinafter: VAD heart pumps) are known for example, which can assist a patient's heart temporarily (bridge-to-recovery) or for preparation for transplantation (bridge-to-transplant) or also permanently (destination therapy). Here, it is important and of great interest to detect and evaluate both the residual cardiac function of the patient's heart and the operating behaviour of the pump.

Previously, it was known to determine variables, such as the differential pressure generated by the pump and the flow rate of the blood delivered by the pump, on the basis of operating parameters of the pump, for example the motor current, the speed and the bearing performance and/or the axial positional shift of a pump rotor in a loaded bearing. On the basis of reference values or calculation models, it is possible to at least partially determine the residual cardiac function from the flow rate delivered by the blood pump. Physiological parameters of the patient's heart can thus be ascertained for diagnosis and therapy purposes, and the operation of the pump can also be optimised and/or controlled. In addition, disturbances at the pump itself, such as thrombus formation or flow abnormalities, can be detected by certain measurements.

Document WO 2015/040221 A2 discloses a blood pump comprising pressure sensors on the pump inlet and outlet. There, use of the detected measured pressure values in order to determine further variables is also described.

The corresponding required operating parameters of the pump are not available in all pump types without difficulty and with the desired accuracy, the desired temporal resolution, the desired sensitivity, or the desired accessibility. For this reason, the object of the present invention is to further develop VAD heart pumps and a corresponding operating method for a VAD heart pump in such a way that parameters related to the pressure of the blood to be delivered can be determined and processed as easily and reliably as possible.

Accordingly, the invention relates to a blood pump and a method for operating a VAD (ventricular assist device) heart pump, in which method a time-dependent pressure of the blood to be delivered is measured directly by means of at least one pressure sensor arranged on the pump inlet and/or on the pump outlet and in addition the temperature of the blood to be delivered is detected by means of at least one temperature sensor.

It should be expressly highlighted that the present invention relates both to a blood pump itself and to a control unit for operating a pump of this kind, an operating method and/or control method for the pump, as well as a computer program product designed to instruct and control a blood pump or control unit accordingly. The term “pump” in this respect can include the peripheral elements, such as a control unit, power supply and display and communication elements.

The method steps specified hereinafter all relate to the above subjects.

By means of the direct measurement by means of a sensor of the time-dependent pressure of the blood to be delivered, the pressure values can be measured with the accuracy, frequency and reproducibility necessary for the particular purpose. The temperature dependency of the other variables, for example the viscosity of the blood, can also be taken into consideration by the temperature measurement. Corresponding sensors are commercially available. They can output the pressure values by means of electrical signals. A pressure sensor of this kind and also a temperature sensor can also additionally comprise a microcontroller for preprocessing the measured values.

The arrangement on the pump inlet or outlet can be provided in such a way that the pressure sensor is directly fixed to the pump. One or more pressure and/or temperature sensors can be provided in a pump inlet or outlet connection piece or on a connection piece of this kind. Connection pieces of this kind are rigid and inflexible, and therefore the operation of the sensor, even at a certain distance from the pump, reflects the pressure directly at the pump. The chosen wording “on the pump inlet and/or pump outlet” is to be understood in this sense.

An accordingly reliable and direct detection of the pressure values and temperature values allows the use for diverse control methods for operation of the pump and also for the determination of physiological values of the patient incorporated indirectly or directly in the control of the pump. The temperature sensors can be arranged in or on the pressure sensors. By taking into account the temperature, a compensation of the temperature influence on the pressure measurement can be implemented. The pressure and temperature sensor can also both be connected to a common microcontroller.

An advantageous embodiment of the method can provide that one or more of the following parameters is/are determined continuously:
the rate of change of the measured pressure per unit of time,
the maximum and/or minimum of the rate of change of the measured pressure within a time period, in particular within one or more cardiac cycles,
the maximum and/or minimum of the time profile of the measured pressure within a time period, in particular within
the heart rate and/or arrhythmias by the profile of the detected pressure values,
the temperature of the delivered blood.

In one embodiment it can be provided that at least one temperature sensor is provided, wherein the temperature sensor is connected to the blood pump and in particular is positioned on the pump inlet on the pump outlet. The temperature passes via the blood-conducting parts to the sensor. The sensor is located for example within the blood pump housing. The best possible thermal coupling to the blood is favourable. By means of the temperature detection directly in the vicinity of the region where the pressure measurement(s) is/are also performed, it is ensured that an incorporation of the temperature values in subsequent calculation or control operations allows a consideration of the dependencies of other variables on the temperature correctly and reliably. This is important for example because a constant temperature of the delivered blood over its entire delivery path cannot be assumed.

It can also be provided that a first temperature sensor is provided on the pump inlet and a second temperature sensor is provided on the pump outlet. A heating of the blood as it passes through the pump, for example as a result of the heating effect of a drive motor or an active magnetic bearing, can thus be taken into consideration.

A further embodiment can provide that at least one temperature sensor is connected to a pressure sensor, in particular is integrated therein. The temperature and pressure sensors can in this way be accommodated in a space-saving manner and can be installed and fixed jointly in a simple way.

For an expedient consideration of at least two measured temperature values, it can be provided that, with regard to the delivery direction of the blood, a first temperature sensor is arranged within the blood pump upstream of the drive motor of the pump and a second temperature sensor is arranged within the pump downstream of the drive motor.

A further embodiment can provide that a pressure sensor is provided externally on the housing of the blood pump in the region that, in the implanted state, protrudes into the thorax and is not acted on by the pressure of the delivered blood. The pressure values measured outside the circulatory system can be taken into consideration advantageously at the time of the evaluation of the pressure values measured in the bloodstream. A pressure value of this kind can be determined advantageously outside the patient's body (atmospheric pressure); however, what is even better is the consideration of the pressure in the thorax, which is subject to the fluctuations by the activities of the respiratory system, which in turn are also affected by the pressure values measured in the blood flow. These interfering external pressure influences can be ascertained and taken into consideration.

Accordingly, it can also be provided that the blood pump is designed, when determining one or more pressure values of the blood to be delivered, to take into consideration an atmospheric pressure detected outside the patient's body and/or a pressure detected by means of the pressure sensor in the patient's thorax.

A further advantageous embodiment provides that the change in rate of the measured pressure per unit of time is determined continuously and the maximum and/or the minimum of this change-related variable is ascertained within a time period. In particular, the change in rate is ascertained within a cardiac cycle. The change in rate of the measured pressure is to be understood to mean the first time derivative of the pressure values. The maximum of the first derivative of the pressure after the time period in the ventricle is used to ascertain the contractility of the heart. Since the pressure prevailing in the ventricle can also be determined from the pressure measured directly at the pump inlet, the maximum and the minimum of the time derivative of the pressure in the ventricle can be determined by the values measured by the pressure sensor.

It can additionally be provided that the preload and afterload of the heart are determined from the measured pressure values over more than one period of the cardiac rhythm.

In the next step it can then be provided that the contractility of the heart is ascertained from the measured and determined variables.

In order to carry out the corresponding evaluations in respect of the contractility, see the article by Sarazan, Kroehle and Main, Left ventricular pressure, contractility and dP/dt(max) in nonclinical drug safety assessment studies, Journal of Pharmacological and Toxicological Methods, 2012.

Contractility is a key parameter of residual cardiac activity and can give an indication of any changes to the capacity of the patient's heart. Conclusions can thus also be drawn with regard to a possible weaning of the patient from the heart pump assistance. An improvement or recovery of contractility is a precondition for weaning. An ascertained indicator of contractility can therefore be output and/or used for the initiation of a weaning process or for the monitoring of the success of a weaning process.

It can also be provided that the heart rate and/or arrhythmia are determined by the course of the detected pressure values. In addition, the amplitude and/or pulsatility of the ventricle pressure can be determined from the measured and determined variables.

A further advantageous embodiment provides that the pressure measured at the pump inlet between the pump and the ventricle is compared continuously to a threshold value which is lower than the pressure values occurring during normal operation, and that an aspiration event is identified in the event that the threshold is undershot, and in particular the pump capacity is immediately reduced. Here, the threshold value can be dependent on a currently detected speed of the pump.

A possible disturbance during the operation of a heart assistance pump in the form of a VAD pump lies in that the intake connection piece of the pump, which protrudes into a ventricle, can become firmly attached to a heart wall. If the aspirating inlet cannula of the pump is moved to within a certain distance of a wall of the heart as a result of a movement of the patient or due to other circumstances (for example low ventricle filling), the negative aspiration pressure causes a further aspiration at the tissue of the heart wall.

In the past, aspiration states of this kind were detected by monitoring the differential pressure across the pump and the operating parameters of the pump. However, a detection of this kind is not always easy, since events other than aspiration can also result in corresponding short-term pressure fluctuations. In addition, the parameters used for the aspiration detection are not direct measured variables, but instead indirect measured variables. With the aid of a direct pressure measurement by means of a pressure sensor, however, a negative pressure can be discovered and identified with much greater certainty. If a pressure below a certain pressure limit not undershot during normal operation is reached as threshold, it can be concluded with great certainty that an aspiration event is present or imminent in the event that the threshold is undershot, since such a low pressure cannot occur other than as a result of aspiration. Aspiration can thus be identified already prior to the complete closure of the inlet cannula and can therefore be avoided preventatively. In order to remedy the undesirable state, the pump capacity can be temporarily reduced, so that the intake end can detach from the heart wall.

A further advantageous embodiment can provide that the pressure of the blood to be delivered is measured at the inlet and at the outlet of the VAD pump by means of two pressure sensors, that a target value for the absolute pressure difference between the inlet and the outlet of the pump is predefined, and that the pump capacity is controlled in such a way that the target value is reached. By means of the control of a pressure difference of this kind across the pump, a certain virtual flow resistance across the pump can be set artificially, which can extend as far as a complete blocking of the pump and thus can simulate the closure of a valve within the pump path. The blood delivered by the residual cardiac activity of the heart is then ejected exclusively by the aorta. Certain conditions that support the residual cardiac activity can be created hereby for the residual cardiac activity. The control of a certain differential pressure can also be used for the taking of physiological measurements at the heart.

The invention can also be configured advantageously in such a way that the pressure of the blood to be delivered is determined at the inlet and at the outlet of the VAD pump by means of two pressure sensors and on this basis the actual pressure difference across the pump is determined, a theoretical value of the pressure difference is ascertained on the basis of detected operating parameters of the pump by comparison with reference values and is compared with the actual value of the pressure difference, and in the event of any deviations the presence of a disturbance of the pump is signalled.

By means of the comparison of the pressure difference which in theory ought to be reached in accordance with the operating parameters of the pump, the pump capacity or the torque of the pump rotor, the speed, the rotor position within the magnetic bearing and other relevant variables with the actually attained and directly measured pressure difference, the capacity of the pump can be determined in comparison to a reference value, for example when the pump is started. If the capacity of the pump lies below a reference or initial value, this can indicate a deterioration of the pump geometry, deformation, thrombus formation or the like, or the creation of a stationary flow in the form of a vortex or a similar phenomenon that inhibits the flow of the blood through the pump. In addition, bearing damage can also lead to a reduced capacity of the pump of this kind. States of this kind can be directly confirmed and signalled by the aforesaid comparison, so that the pump can be serviced or replaced.

A further advantageous embodiment of the invention can provide that the heart rate is determined by the course of the detected pressure values. Furthermore, arrhythmias of the heart rate can be ascertained on this basis.

An advantageous method can additionally provide that the cardiac output HZV is determined from the detected heart rate HR, the time difference ED between the time t₁ (dP/dtₘₐₓ) at which the speed of the change in pressure reaches its maximum and the time t₂ (dP/dtₘᵢₙ) at which the speed of the change in pressure reaches its minimum, the pressure P₁ₛₜ at the time of the maximum blood flow and the pressure P_{ES} at the time t₂, in accordance with the formula

$$HZV = HR \frac{( {P}_{1 st} - {P}_{ES} ) ED}{2 {Z}_{C}} .$$

The described determination of the cardiac output is based on a linear approximation, which assumes that the flow rate through the pump as a first approximation is proportional to the ventricular pressure. This model leaves elasticity effects of the circulatory system out of consideration.

It can additionally be provided that the pressure profile in the aorta is determined from the ventricle pressure profile, the differential pressure across the pump and/or the time-dependent pressure drop across the outlet cannula, and from this the ejection fraction is determined. Since the ejection fraction can be determined from the cardiac output under consideration of the end-diastolic residual volume of the heart, the ejection fraction can thus be determined with use of the continuously detected pressure values in the ventricle/at the pump inlet.

The subject of the present application is additionally a heart pump device comprising a VAD heart pump, a control unit, a temperature sensor and at least one pressure sensor on the pump inlet and/or on the pump outlet for carrying out the method.

The invention also relates to a method for operating a heart pump, in which method a time-dependent pressure of the blood to be delivered is measured directly by means of at least one pressure sensor arranged on the pump inlet and/or on the pump outlet, and in which the temperature of the delivered blood is additionally detected by a temperature sensor arranged in or on the blood pump.

The invention will be presented and explained hereinafter on the basis of exemplary embodiments in figures of a drawing, in which

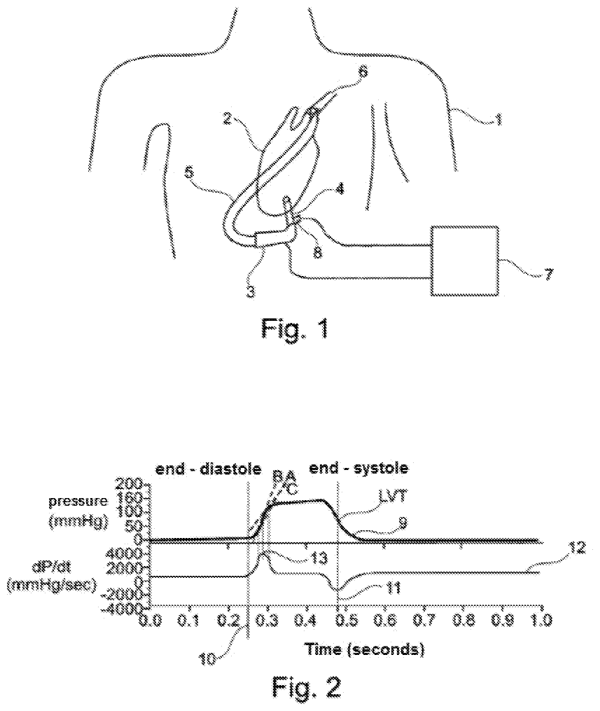

FIG. 1 schematically shows the arrangement of a heart assistance pump in a patient's body,

FIG. 2 shows a detected pressure profile in the region of the pump,

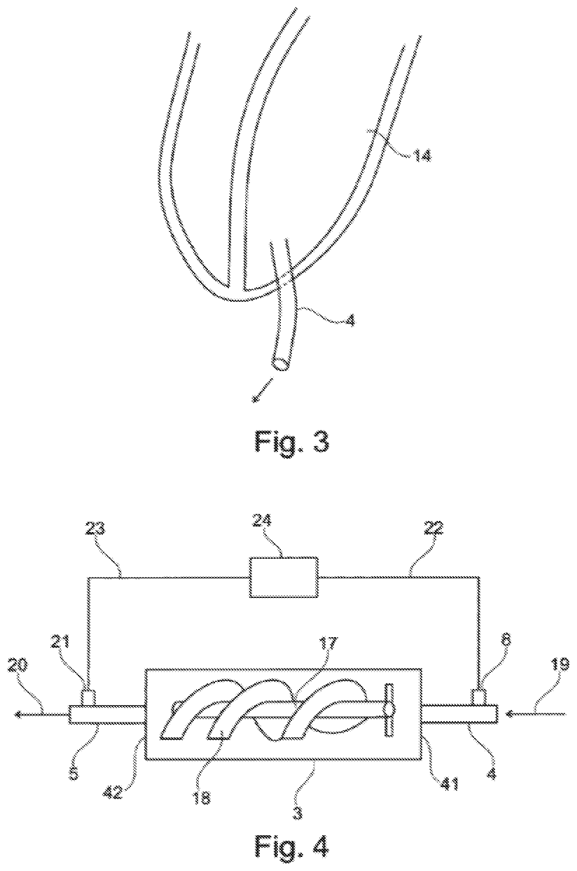

FIG. 3 shows the intake end of a cannula connected to a heart assistance pump in a ventricle,

FIG. 4 shows a heart assistance pump with two pressure sensors,

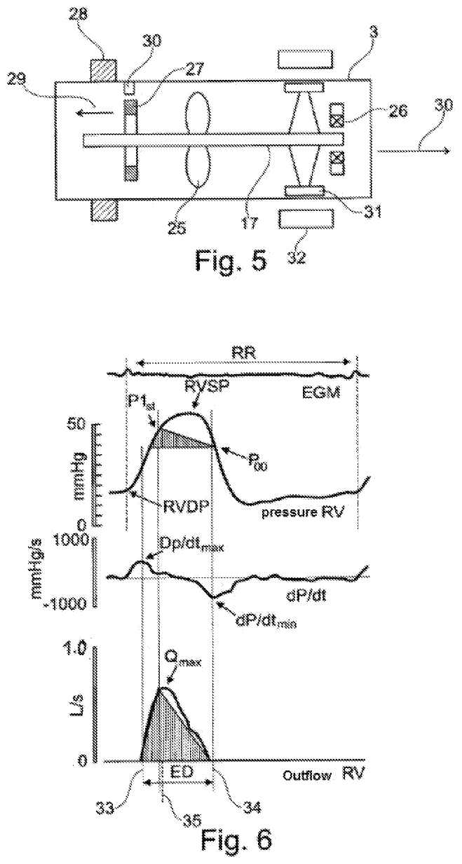

FIG. 5 shows the basic functional elements of a typical heart assistance pump,

FIG. 6 shows three graphs of a model for determining the cardiac output on the basis of ventricular pressure measurements,

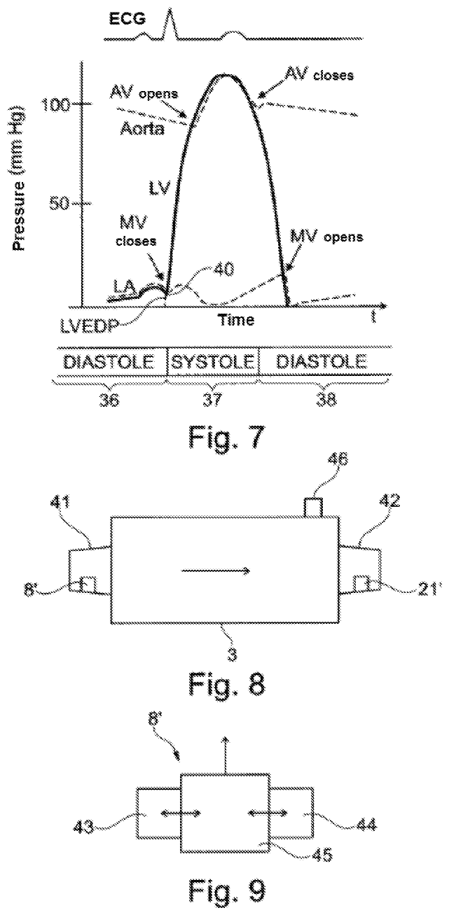

FIG. 7 shows a graph showing the development of the ventricular pressure over two diastoles and an intermediate systole,

FIG. 8 schematically shows a blood pump having combined pressure and temperature sensors on each of the inlet and outlet, and

FIG. 9 shows the schematic structure of a combined sensor.

FIG. 1 schematically shows the upper body 1 of a patient with a schematic depiction of the heart 2 and of a heart pump 3. The heart pump 3 is connected via a cannula 4 on the intake side to a ventricle of the heart 2 and at that point aspirates the blood that is to be delivered via a pump inlet. The blood is delivered through the pump 3, through the outlet cannula 5 to the aorta 6 and is introduced into the aorta. The heart 2 of the patient, besides the pump function of the heart pump 3 itself, can also deliver blood into the aorta by contraction of the heart muscle within the scope of what is known as the ‘residual cardiac function’, such that the heart 2 and the heart pump 3 work in parallel and in a manner assisting one another.

The heart pump 3 is connected to a control unit 7, which supplies the heart pump with power and actuates a control method accordingly. The control unit 7 is additionally connected to a pressure sensor 8, which is arranged in the bloodstream in the region of the intake cannula 4 on the pump inlet 41 and signals the pressure of the blood to be delivered in the intake cannula. The pressure sensor 8 communicates pressure values by means of electrical signals to the control unit 7. By means of the feedback regarding the actual pressure value between the ventricle and the pump 3, the various advantageous features of embodiments according to the invention can be realised.

FIG. 2 shows, in a graph, on the horizontal x-axis a time scale in seconds and in the upper part a curve, in which the pressure of the blood, more specifically the pressure difference relative to the atmospheric pressure, is plotted over time. The pressure curve is denoted by 9.

In a first phase to the time 10 the pressure remains at a low and practically constant level, wherein the time 10 represents the end of the diastole. The pressure then rises by contraction of the heart, so as to then drop again at the end of the systole, i.e. at the time 11. In the figure, three tangents A, B and C are placed against the curve 9 in the region of the pressure rise and show the different times of different gradients of the pressure profile.

In accordance with the method according to the invention the liquid pressure can be determined with high temporal resolution, so that the changes over time of the pressure, i.e. the gradients of the pressure curve, can also be ascertained with corresponding resolution.

In the graph of FIG. 2 values for the time derivative of the pressure are shown in the lower part below the pressure curve 9 in a curve 12. It can be seen that, after the time 10, not only does the pressure initially rise, but also the rate of the pressure rise, up to a maximum 13. This value corresponds to dP/dtₘₐₓ, i.e. the maximum of the first pressure derivative. This maximum lies at the end of the diastole. In the time region between the time 10 and the time 11, a phase with low rates of change of the pressure starts once the maximum has been reached, wherein at the end of the systole the value for dP/dt reaches a minimum dP/dtₘᵢₙ as the second time 11 is approached.

The value dP/dtₘₐₓ is used, according to Sarazan, Kroehle and Main, 2012, to estimate the contractility during the residual cardiac function. In order to be able to ascertain the contractility in accordance with the aforesaid method, at least also the preload of the ventricle must be recorded. In a simplified conception the end-diastolic pressure in the heart can be understood to be the preload. Reference is made to the explanations for FIG. 7 in this respect.

FIG. 3 serves to depict the phenomenon of aspiration in the ventricle. An intake cannula 4 is shown in schematic form. By means of aspiration at the heart wall, the intake opening can be closed at least in part and thus blocked.

By means of the direct pressure measurement in the region of the intake cannula 4 or the inlet opening of the pump 3, the pressure development can be tracked with high temporal resolution, so that a drop in pressure can be responded to quickly and the capacity of the pump can be reduced. The intake opening of the cannula 4 can thereupon be detached from the heart wall. The direct measurement of pressure values by means of a pressure sensor not only allows measurements of high temporal resolution, but also unambiguous interpretations of the pressure values and a reliable response in the event that correspondingly set threshold values are undershot. Aspiration can thus be identified already before the complete closure of the inlet cannula, and therefore aspiration can be avoided preventatively.

FIG. 4 schematically shows a longitudinal section through a heart pump 3 with a hub 17 and delivery elements in the form of helical blades 18. An inlet cannula/intake cannula 4 is shown, through which blood is aspirated in the direction of the arrow 19, and an outlet cannula 5 is shown, which is connected to the pump outlet 42 and by means of which the blood is ejected in the direction of the arrow 20. In the shown exemplary embodiment a first pressure sensor 8 is arranged in the intake cannula 4, whereas a second pressure sensor 21 is arranged in the outlet cannula 5. The two pressure sensors can also be arranged directly on the respective ends of the hub 17. Both pressure sensors 8, 21 are connected by means of signal lines 22, 23 to a control device 24 for controlling the pump 3.

In FIG. 5 the mechanical structure, the bearing support, and the drive of the pump 3 are shown schematically. In principle, diverse pump constructions can be employed within the scope of the invention, for example pure axial-rotor pumps, and also axial/radial-rotor pumps. The hub of the rotor is denoted by 17 in FIG. 5, wherein a delivery element 25 of the rotor in the form of a delivery blade is shown merely by way of representation. The delivery elements are each fixedly connected to the hub 17 of the rotor. The hub 17 is supported in a first radial bearing 26 in the radial direction. The radial bearing 26 for example can be formed as a hydrodynamic bearing. In addition, at the opposite end of the hub 17, said hub is connected to a magnetic ring 27, which cooperates with a stationary magnetic ring 28 in order to form an axial/radial bearing. An axial repulsion between the magnetic rings 27, 28 causes axial forces to be intercepted that press the rotor in the direction of the arrow 29 on account of the fact that the rotor delivers the blood in the direction of the arrow 30. The axial force on the rotor to be intercepted corresponds in this respect to the reaction force of the blood to be delivered. The repulsive force between the magnetic rings 27 and 28 not only brings about a bearing support in the axial direction, but as appropriate, if there is no additional radial bearing provided, also in the radial direction. The magnetic repulsive force of the stationary magnetic ring 28 is at least partially caused by means of a magnetic field generated by an electromagnet. By appropriate actuation of the coil of the electromagnet, it is possible to control the magnetic bearing in order to keep the axial position of the rotor or the magnetic ring 27 as constant as possible. In order to form a closed control loop, the axial position of the magnetic ring 27 is detected by means of a position sensor 30.

The current strength at which the magnetic bearing 28 must be actuated in order to apply the axial force for holding the rotor in the axial direction can be used to calculate the pressure difference generated by the pump. The torque acting on the rotor as well as the speed of the rotor can also be used for this purpose.

The rotor is driven in rotation by means of an electromotive drive, wherein a rotor part 31 comprises permanent magnets that are driven in the field of a stator 32 in the peripheral direction. The stator 32 has windings, which can be actuated within the scope of the actuation of a brushless motor, for example by pulse width-modulated signals. The monitoring of the stator currents allows a determination of the torque.

On the basis of FIG. 6 a model for calculating/estimating the cardiac output will be described. The cardiac output indicates the volume of blood delivered via the aorta into the body's circulation system. By determining the pump parameters, merely the flow delivered by the pump can be ascertained. By consideration of the pressure and change in pressure variables in the region of the heart, the degree of assistance or the cardiac output of the heart itself can be ascertained. This is performed on the basis of what is known as pulse contour analysis. Herein, the continuous profile of the arterial pressure is determined, and from this the cardiac output is determined. Herein, a linear model, i.e. a linear relationship between the ventricular pressure and the delivered blood volume, is used as a basis. The delivered cardiac output is estimated from the following formula:

$$HZV = HR \frac{( {P}_{1 st} - {P}_{ES} ) ED}{2 {Z}_{C}}$$

Here, HZV is the estimated cardiac output, HR is the heart rate, Z_{C} is the patient-specific impedance of the aorta, and the variables P₁ₛₜ, P_{ES} and ED are given from FIG. 6. The basis here is provided by the scientific findings of Valsecchi et al., Cardiac output derived from left ventricular pressure during conductance catheter evaluations: an extended model flow method, Journal of Clinical Monitoring and Computing, 2007 and Karamanoglu, Estimation of cardiac output in patients with congestive heart failure by analysis of right ventricular pressure waveforms, Biomedical Engineering Online, 2011. To this end, the pressure profile of the ventricular pressure for example from the end of one diastole to the end of the next diastole is shown in the upper part of FIG. 6. The ventricular pressure increases here up to a maximum and then drops at the end of the systole.

The same period of time is shown in the middle depiction of FIG. 6 on the basis of a graph, in which the changes in the ventricular pressure are shown as a time derivative of the detected pressure value. Here, the time 33 is the time at which the maximum dP/dtₘₐₓ is reached in the middle depiction of FIG. 6. Reference sign 34 denotes the time at which the minimum dP/dtₘᵢₙ of the change in pressure is reached in the middle graph of FIG. 6. The difference between the times 33 and 34 is the period ED. P_{ES} denotes the pressure value reached at the time 34.

In the lower graph of FIG. 6 the flow through the aorta in litres per second is plotted over the time between the times 33 and 34. A maximum triangle is inscribed within the curve denoting the flow. The upper tip of the triangle defines the time 35 at which the pressure value P₁ₛₜ is detected. All variables that are to be used in the above-mentioned equation for ascertaining the cardiac output are thus defined.

On the basis of FIG. 7, the method for ascertaining the preload and after-load is described on the basis of a continuous absolute pressure measurement. The graph shows the pressure profile, wherein the pressure is plotted on the y-axis and the time is plotted on the horizontal x-axis. With regard to the time, a first diastole 36, a subsequent systole 37, and a subsequent further diastole 38 are considered. At the end of the first diastole 36, the pressure first passes through a relative maximum 39, before a sharp V-shaped minimum is passed through and the pressure then rises significantly in the systole. The pressure value of the minimum 40 constitutes the left-ventricular end-diastolic pressure and is therefore a measure for the preload. As described above in conjunction with FIG. 2, this value can be used inter alia to ascertain the cardiac output.

FIG. 8 schematically shows the blood pump 3 with a pump inlet 41 and a pump outlet 42. A first combined pressure/temperature sensor 8′ is provided at the pump inlet, in or on the pump tube, and a second combined pressure/temperature sensor 21′, which is structurally identical to the first sensor 80′, but can also be embodied differently therefrom, is provided at the pump outlet 42. A pressure-measuring sensor 46 is arranged externally on the housing of the pump 3 and is provided to measure the thoracic pressure and transmits the measured values to a control unit of the pump.

FIG. 9 schematically shows a pressure sensor element 43 within a pressure/temperature sensor 8′, a temperature sensor element 44 mechanically connected directly or by means of a common supporting part to said pressure sensor element, and a microcontroller 45, which is connected at least electrically, in particular also mechanically, to both sensor elements. This microcontroller also has a communication function and can conduct or send preprocessed measurement variables to a control unit of the pump.

In this regard, three aspects of the invention should be emphasised, which can each individually constitute an invention without dependency reference: a method for operating a VAD heart pump, in which method the pressure of the blood to be delivered is measured at the inlet and at the outlet of the VAD pump by means of two pressure sensors and from this the actual pressure difference is determined, a theoretical value of the pressure difference is ascertained on the basis of detected operating parameters of the pump by comparison with reference values and said theoretical value is compared with the actual value of the pressure difference, and in the event of any deviations the presence of a disturbance of the pump is signalled; a method for operating a VAD heart pump, in which method absolute pressure values of the time-dependent pressure of the blood to be delivered are detected by a pressure sensor at the inlet of the pump or between the inlet of the pump and the ventricle and the heart rate is ascertained by the profile of the detected pressure values; and a method for operating a VAD heart pump, in which method the cardiac output HZV is determined from the detected heart rate HR, the time difference ED between the time t₁ (dP/dtₘₐₓ) at which the speed of the change in pressure reaches its maximum and the time t₂ (dP/dtₘᵢₙ) at which the speed of the change in pressure reaches its minimum, the pressure P₁ₛₜ at the time of the maximum blood flow, and the pressure P_{ES} at the time t₂, in accordance with the formula

$$HZV = HR \frac{( {P}_{1 st} - {P}_{ES} ) ED}{2 {Z}_{C}} .$$

Here, the independent invention can also be configured such that the pressure profile in the aorta is determined from the ventricular pressure profile, and from this the end-diastolic volume of the heart is determined, and in particular the ejection fraction is determined with use of this value.

The following aspects can represent independent inventions, in each case individually or in combination with one another or with the claims of this application:
Aspect 1: A blood pump, in particular a VAD (ventricular assist device) heart pump (3), which is designed to directly measure a time-dependent pressure of the blood to be delivered by means of at least one pressure sensor (8, 21) arranged on the pump inlet (4, 41) and/or on the pump outlet (5, 42).
Aspect 2: The blood pump according to aspect 1, designed in such a way that one or more of the following parameters is/are continuously determined:
   the rate of change of the measured pressure per unit of time,
   the maximum and/or minimum of the rate of change of the measured pressure within a time period, in particular within one or more cardiac cycles,
   the maximum and/or minimum of the time profile of the measured pressure within a time period, in particular within one or more cardiac cycles,
   the heart rate and/or arrhythmias by the profile of the detected pressure values.
Aspect 3: The blood pump according to aspect 1, designed in such a way that the rate of change of the measured pressure per unit of time is continuously determined and the maximum and/or minimum of this change variable within a period of time is determined, in particular within a cardiac cycle.
Aspect 4: The blood pump according to aspect 2 or 3, designed in such a way that the preload and/or the afterload of the heart is additionally determined from the measured pressure values over more than one period of the cardiac rhythm.
Aspect 5: The blood pump according to aspect 2, 3 or 4, designed in such a way that the contractility of the heart is determined from the measured and determined variables.
Aspect 6: The blood pump according to aspect 2, 3 or 4, designed in such a way that the indicator of the contractility is used for the initiation of a weaning process and/or the monitoring of the success of a weaning process.
Aspect 7: The blood pump according to aspect 1 or any one of the following aspects, designed in such a way that the heart rate and/or arrhythmias is/are determined by the profile of the detected pressure values.
Aspect 8: The blood pump according to aspect 2 or any one of the following aspects, designed in such a way that the amplitude and/or pulsatility of the ventricular pressure is determined from the measured and determined variables.
Aspect 9: The blood pump according to aspect 1 or any one of the following aspects, designed in such a way that the pressure measured at the pump inlet (4, 41) between the pump (3) and the ventricle is compared continuously with a threshold value that is lower than the pressure values occurring during normal operation, and, in the event that the threshold is undershot, an aspiration event is identified and in particular the pump capacity is immediately reduced, wherein the threshold value is dependent in particular on the speed of the pump.
Aspect 10: The blood pump according to aspect 1 or any one of the following aspects, designed in such a way that the pressure of the blood to be delivered is measured at the inlet and at the outlet of the VAD pump (3) by means of two pressure sensors (8, 21), a target value for the absolute pressure difference between the inlet and the outlet of the pump (3) is predefined, and the pump capacity is controlled in such a way that the target value is reached.
Aspect 11: The blood pump according to aspect 1 or any one of the following aspects, designed in such a way that the pressure of the blood to be delivered is measured at the inlet and at the outlet of the VAD pump (3) by means of two pressure sensors (8, 21) and the actual pressure difference is determined on this basis, a theoretical value of the pressure difference is determined on the basis of detected operating parameters of the pump (3) by comparison with reference values and is compared with the actual value of the pressure difference, and in the event of any deviations the presence of a disturbance of the pump is signalled.
Aspect 12: The blood pump according to aspect 1 or any one of the following aspects, designed in such a way that the cardiac output (HZV) is determined from the detected heart rate HR, the time difference ED between the time t₁ (dP/dtₘₐₓ) at which the speed of the change in pressure reaches its maximum and the time t₂ (dP/dtₘᵢₙ) at which the speed of the change in pressure reaches its minimum, the pressure P₁ₛₜ at the time of the maximum blood flow and the pressure P_{E} at the time t₂, in accordance with the formula

$$HZV = HR \frac{( {P}_{1 st} - {P}_{ES} ) ED}{2 {Z}_{C}} .$$

Aspect 13: The blood pump according to aspect 10, designed in such a way that the pressure profile in the aorta is determined from the ventricular pressure profile, the pressure difference across the pump, and the time-dependent pressure drop, and from this the ejection fraction is determined.
Aspect 14: The blood pump according to any one of the preceding aspects, containing a control device (7, 24) and at least one pressure sensor (8, 21) on the pump inlet (41) and/or on the pump outlet (42).
Aspect 15: A method for operating a heart pump according to any one of the preceding aspects, in which a time-dependent pressure of the blood to be delivered is measured directly by means of at least one pressure sensor arranged on the pump inlet and/or on the pump outlet.

1.-21. (canceled)

22. A blood pump comprising:
a pump inlet;
a pump outlet;
at least one pressure sensor; and
at least one temperature sensor,
wherein the blood pump is configured to directly measure a time-dependent pressure of blood to be delivered by means of the at least one pressure sensor arranged on the pump inlet, the pump outlet, or both the pump inlet and the pump outlet, and wherein the blood pump is further configured to detect a temperature of the blood to be delivered by means of the at least one temperature sensor.

23. The blood pump according to claim 22, wherein the blood pump is configured to continuously determine one or more of the following parameters:
a rate of change of the measured time-dependent pressure per unit of time,
a maximum or a minimum or both a maximum and a minimum of the rate of change of the measured time-dependent pressure within a time period of one or more cardiac cycles,
a maximum or a minimum, or both a maximum and a minimum of a time profile of the measured time-dependent pressure within the time period,
a heart rate or arrhythmias, or both a heart rate and arrhythmias, by a profile of detected pressure values,
the temperature of the blood.

24. The blood pump according to claim 22, wherein the at least one temperature sensor is connected to the blood pump and is positioned on the pump inlet or on the pump outlet.

25. The blood pump according to claim 22, wherein the at least one temperature sensor comprises a first temperature sensor provided on the pump inlet and a second temperature sensor provided on the pump outlet.

26. The blood pump according to claim 22, wherein the at least one temperature sensor is connected to the at least one pressure sensor and is integrated therein.

27. The blood pump according to claim 22, wherein the at least one temperature sensor comprises a first temperature sensor arranged within the blood pump upstream of a drive motor of the blood pump, and a second temperature sensor arranged within the blood pump downstream of the drive motor.

28. The blood pump according to claim 22, wherein the at least one pressure sensor comprises a pressure sensor provided externally on a housing of the blood pump in a region that, in an implanted state of the blood pump, protrudes into a thorax of a patient and is not acted on by a pressure of the blood to be delivered.

29. The blood pump according to claim 22, wherein the at least one pressure sensor is configured to detect pressure in a thorax of a patient, and the blood pump is configured to determine one or more pressure values of the blood to be delivered based on the pressure detected in the thorax or an atmospheric pressure measured outside the patient's body, or both the pressure detected in the thorax and the atmospheric pressure measured outside the patient's body.

30. The blood pump according to claim 22, wherein the blood pump is configured to continuously determine, during a cardiac cycle, a rate of change of the measured time dependent pressure per unit of time and determine, within a period of time, a maximum of the rate of change, or a minimum of the rate of change, or the maximum and the minimum rate of change.

31. The blood pump according to claim 23, wherein the blood pump is configured to determine a preload of a heart of a patient, or an afterload of the heart of the patient, or both the preload and the afterload of the heart of the patient based on the measured time-dependent pressure of blood to be delivered over more than one period of a cardiac rhythm of the heart.

32. The blood pump according to claim 23, wherein the blood pump is configured to determine contractility of a heart of a patient based on measured and determined variables.

33. The blood pump according to claim 32, wherein the blood pump is configured to provide an indicator of the contractility of the heart, the indicator used for initiation of a weaning process, or monitoring success of the weaning process, or both initiation and monitoring success of the weaning process.

34. The blood pump according to claim 22, wherein the blood pump is configured to determine a heart rate, or arrhythmias, or both the heart rate and the arrhythmias, based on a profile of values of the measured time dependent pressure of the blood.

35. The blood pump according to claim 23, wherein the blood pump is configured to determine, based on measured and determined variables, an amplitude of a ventricular pressure, or pulsatility of the ventricular pressure, or both the ventricular pressure and the pulsatility of the ventricular pressure.

36. The blood pump according to claim 22, wherein the blood pump is configured to continuously compare a pressure measured at the pump inlet to a threshold value that is lower than pressure values occurring during normal operation, wherein the pump inlet is between the blood pump and a ventricle of a patient's heart, and the blood pump is further configured, in response to the pressure measured at the pump inlet being less than the threshold value, to identify an aspiration event and immediately reduce a pump capacity of the blood pump, wherein the threshold value is dependent on a speed of the blood pump.

37. The blood pump according to claim 22, wherein the at least one pressure sensor comprises a first pressure sensor arranged on the pump inlet to measure a pressure of the blood to be delivered at the pump inlet and a second pressure sensor arranged on the pump outlet to measure a pressure of the blood to be delivered at the pump outlet, wherein a target value for an absolute pressure difference between the pump inlet and the pump outlet is predefined, and wherein the blood pump is configured to control a pump capacity such that the target value is reached.

38. The blood pump according to claim 22, wherein the at least one pressure sensor comprises two pressure sensors, wherein the blood pump is configured to measure, with the two pressure sensors, a pressure of the blood at the pump inlet and a pressure of the blood at the pump outlet, wherein the blood pump is configured to determine an actual pressure difference based on pressure measured by each of the two pressure sensors, and a theoretical pressure difference based on comparison of detected operating parameters of the blood pump with reference values, wherein the blood pump is further configured to compare the theoretical pressure difference with the actual pressure difference, and in response to any deviations, the blood pump is configured to signal the presence of a disturbance of the blood pump.

39. The blood pump according to claim 22, wherein the blood pump is configured to determine a cardiac output (HZV) based on a detected heart rate HR in accordance with a formula
$$HZV = HR \frac{( {P}_{1 st} - {P}_{ES} ) ED}{2 {Z}_{C}} ,$$
wherein ED is a time difference between a time t₁ (dP/dtₘₐₓ), at which a speed of change in pressure reaches a maximum, and a time t₂ (dP/dtₘᵢₙ), at which the speed of change in pressure reaches a minimum, P₁ₛₜ is a pressure at the time of the maximum blood flow and P_{ES} is a pressure at the time t₂.

40. The blood pump according to claim 37, wherein the blood pump is configured to determine an ejection fraction from a pressure profile in an aorta of a patient's heart, the pressure profile determined by the blood pump from a ventricular pressure profile, a pressure difference across the blood pump, and a time-dependent pressure drop across an outlet cannula.

41. The blood pump according to claim 22, further comprising a control device.

42. The blood pump of claim 22, wherein the blood pump comprises a VAD (ventricular assist device).

43. A method comprising:
receiving blood at an inlet of a blood pump;
delivering the blood at an outlet of the blood pump;
directly measuring a time-dependent pressure of the blood with at least one pressure sensor, the at least one pressure sensor arranged on the pump inlet or on the pump outlet or on both the pump inlet and the pump outlet; and
detecting a temperature of the blood with a temperature sensor arranged in or on the blood pump.
